(19) United States
(12) Patent Application Publication
(10) Pub. No.: US 2013/0204296 A1
(43) Pub. Date: Aug. 8, 2013

(54) Absorbable Suture Device

(71) Applicant: Daniel Brauman, Rye Brook, NY (US)

(72) Inventor: Daniel Brauman, Rye Brook, NY (US)

(21) Appl. No.: 13/757,786

(22) Filed: Feb. 2, 2013

Related U.S. Application Data
(60) Provisional application No. 61/595,176, filed on Feb. 6, 2012.

(51) Int. Cl. A61B 17/04

(52) U.S. Cl. CPC A61B 17/0401
USPC 606/232

(57) ABSTRACT

A suture device including one or more sutures and one or more suture retention members, each suture retention member anchoring a portion of a fiber of the one or more sutures. The suture device may be adapted to hold together and approximate skin edges. The suture may be situated both within the skin and subcutaneous tissue. The suture device may be anchored to the undersurface of one or more of the skin and the subcutaneous layer.

### CROSS REFERENCE TO RELATED APPLICATIONS

The application claims the benefit of U.S. Provisional Patent Application Ser. No. 61/595,176 filed Feb. 6, 2012 to Brauman, titled “Absorbable Suture Device”, the entire contents thereof being incorporated herein by reference.

### FIELD

This invention relates generally to an absorbable suture and more particularly to an absorbable suture that is adapted to hold together and approximate skin edges without the need for external sutures or staples.

### BACKGROUND

The skin broadly consists of two layers. The epidermis is a thin outer skin layer. The dermis sits below and attaches to the epidermis and usually forms the bulk of the thickness of the skin of a person. An additional layer that lies below the skin is the subcutaneous fat layer, consisting mostly of fat held within a layer of connective tissue.

After surgery, or when joining portions of the skin is required regardless of the origin of the incision in the skin, use of a suture will typically be necessary. Furthermore, because of ease of use and patient's comfort an absorbable suture is desirable to be used to close the incision as such sutures need not be removed; they simply dissolve away over time.

The vast majority of absorbable sutures and devices are formed of various polymers and collagens. Polymers of glycolic and lactic acids are very frequently used. Other materials such as Polydioxanone, Poliglecaprone 25 and Collagen rich materials are also widely used. Additionally, these sutures may be coated with various substances. The Polymers of glycolic and lactic acids are degraded and absorbed by the biochemical process of hydrolysis. The hydrolysis of these polymer materials is often characterized by a low level of inflammation. Absorption of the suture device begins as a loss of tensile strength of the suture device, and then a loss of mass thereof

A number of prior art suture devices are available. A barbed wire suture (by Covidien, for example), comprises an absorbable suture made of a lactic/glycolic polymer with unidirectional barbs. This wound closure device provides a continuous closure that is fluid tight, and is therefore unable to provide an interrupted suture closure that would allow for drainage through the incision. This device further requires the use of a needle holder and often the placement of surgical knots, which is time consuming.

The inventor of the present invention has determined that a low level of tissue inflammation, and thus the relative inertness of polymers employed in typical absorbable sutures or staples, is one of the main reasons for the use of such polymers as absorbable materials in the absorbable sutures or staples. The inventor of the present invention has further determined that one major drawback of these glycolic/lactic acid polymers is that they do not do well close to the skin surface. The inventor of the present invention has additionally determined that this is because hydrolysis does not proceed well within the relatively dry dermis. Thus, surgical knots which are bulky tend to extrude out of the skin (known as spitting sutures) and may also cause skin abscesses known as stitch abscesses. This is a very common occurrence when such traditional polymer skin sutures are used near the skin surface. Despite these drawbacks, these sutures are the most commonly employed absorbable sutures to approximate skin because of their relative inertness and tensile strength that lasts long enough (i.e. several weeks) for the skin edges to heal and form a strong enough bond before the sutures are fully absorbed.

The inventor of the present invention has further observed that other absorbable sutures may be formed of a collagen material. Such collagen sutures are absorbed through a process known as enzymatic degradation and degrade well within the dermis. This degradation may, however, be accompanied by a pronounced inflammatory tissue response. Additionally, the collagenous sutures (typically known as cat gut and formed of either synthetic or non-synthetic materials) do not possess the tensile strength that the polymers have, and further tend to degrade more quickly. These sutures, therefore, are best employed as rapidly absorbing sutures.

Dermal staples, such as those that may be applied through the use of an Incisive® surgical stapler, comprise thick, absorbable staples, not sutures. These thick staples suffer from the disadvantage that they can only be used in dermis that is thick. During insertion of these staples, the skin must be pulled up high. If the dermis is thin, the process of insertion of these staples may tear through the skin. Once inserted, these staples may still erode through the skin. Such dermal staples further require very precise placement. If the staples are placed too superficially, they may erode through the skin. These staples are typically bulky and thick, take a long time to absorb, and can be palpated through the skin for a long time.

It would be therefore beneficial to provide a suture device that overcomes the drawbacks of the prior art.

### SUMMARY

The inventor of the present invention has determined that hydrolysis does not proceed well within the relatively dry dermis and that it proceeds better in the more moist environment of the subcutaneous fat. Therefore, surgical knots and staples made of glycolic/lactic acid polymers would be expected to be less well tolerated and absorbed in the dermis and better tolerated and absorbed within the subcutaneous tissue. In addition, the inventor of the present invention has determined that foreign bodies such as sutures are better tolerated deeper, further away from the skin surface. Therefore, surgical knots and staples would be expected to be less well tolerated and absorbed in the dermis and conversely better tolerated and absorbed within the subcutaneous tissue. Also, the inventor of the present invention has determined that the more bulky and rigid a foreign body is, the less well it will be tolerated in the skin or in the subcutaneous fat layer. Therefore, reduction in the bulk and rigidity of staples and sutures is preferred especially closer to the outer surface of the skin, and particularly in the dermis.

In order to overcome the problem of stitch abscesses and suture extrusion, which occurs very frequently with absorbable sutures, the inventor of the present invention, an experienced surgeon, has previously successfully modified the absorbable suture technique which has been practiced for at least since the early 1970's. Traditionally, when an absorbable suture is placed, the surgeon places a knot and directs the assistant to cut both suture ends at a length judged to be long enough to prevent the knot from unraveling and short enough to prevent the suture ends from protruding through the incision to the outer surface of the skin. Additionally, cutting the suture ends short is aimed at reducing the amount of suture material/foreign body in the subcutaneous tissue since foreign bodies greatly increase the potential for wound infection.

The inventor of the present invention had determined that the main cause of suture abscesses and suture extrusion/spitting, was the close proximity of the bulky and open-pore knot, to the skin surface as well as the knot impinging on the undersurface of the skin. Therefore, for many years, the inventor has been grasping the suture ends with a clamp and/or needle holder at a length substantially longer, usually at least twice as long as would have been judged by any surgeon skilled in the art. The assistant then cuts the suture ends and the inventor inserts the suture ends into the subcutaneous fat. This directs the knot into the subcutaneous tissue and anchors it there by means of the long suture ends. This novel surgical technique has virtually eliminated the occurrence of suture abscesses and spitting sutures. Additionally, the inventor of the present invention had determined that the mere presence of a foreign body in the subcutaneous tissue greatly increases the likelihood of a wound infection and that bulkier, more rigid sutures or staples especially those with open-pores/interstices, further decrease tissue resistance and increase the risk of wound infections.

The current invention is aimed at utilizing this tested and beneficial concept and providing an apparatus that will have the additional benefits of expediting the placement of absorbable sutures precisely and consistently. Reduction of operative time is greatly beneficial to a positive outcome of any surgery.

In accordance with various embodiments of the invention, an absorbable suture device is provided that includes a suture or sutures secured in place by one or more absorbable retention members. The suture may be formed of an appropriate material, whether it be coated, uncoated, mono or multi filament, or the like. The suture may also be smooth or textured and possess various types of indentations or projections, pores and/or interstices. The retention member or members or other retention features preferably function in place of a surgical knot. The inventive absorbable surgical device is adapted to hold together and approximate the skin edges of an incision without the need of external sutures or staples that need to be removed, cause pain, and leave visible suture marks. In accordance with various embodiments of the present invention, an improved absorbable suture device is provided that expedites placement of an absorbable suture, thus shortening surgery time. The inventive absorbable suture device also provides an interrupted suture closure that allows fluid drainage through the incision, thus reducing swelling and preventing fluid collection that may cause an infection.

Any compatible material may be used, that may be absorbable, partially absorbable or non absorbable, however, and in order to benefit from the advantageous biomechanical properties of glycolic/lactic polymers (tensile strength, strength retention, and minimal inflammation) and minimize the disadvantage of poor and slow absorbability in the dermis, a compound suture device is preferably provided in accordance with one or more embodiments of the invention that combines a suture fiber adapted to be placed within the dermis and solid retention members adapted to be embedded in the subcutaneous fat layer to anchor the suture fiber. The retention member may also be formed of fiber, but is preferably more bulky, and may also or alternatively be provided as more rigid or stiff than the suture fiber or be shaped in a way that will anchor it to the undersurface of the skin in order to maintain a proper anchor for the suture fiber, and to thus retain the suture fiber in place and approximate the everted skin edges.

Both the suture and retention members/devices may also be made of absorbable, non-absorbable or partially absorbable materials

Still other objects and advantages of the invention will in part be obvious and will in part be apparent from the specification and drawings.

The invention accordingly comprises the several steps and the relation of one or more of such steps with respect to each of the others, and the apparatus embodying features of construction, combinations of elements and arrangement of parts that are adapted to affect such steps, all as exemplified in the following detailed disclosure, and the scope of the invention will be indicated in the claims.

### BRIEF DESCRIPTION OF THE DRAWINGS

For a more complete understanding of the invention, reference is made to the following description and accompanying drawings, in which:

FIG. 1 depicts a suture device constructed in accordance with a preferred embodiment of the invention; and

FIG. 2 depicts the suture device of FIG. 1 situated within tissue layers.

### DETAILED DESCRIPTION OF THE PREFERRED EMBODIMENTS

Various embodiments of the present invention operate on a number of basic principles, including but not limited to:
1. Hydrolysis is thought to proceed better in the subcutaneous tissue than within the skin.
2. Depth of foreign body from surface. The deeper a foreign body is relative to the outer surface of the skin, the better the foreign body may be tolerated and the less chance that it will be extruded. Moreover, even a deeply placed non-absorbable suture may be better tolerated than a superficially placed absorbable suture.
3. Bulk. The smaller the volume/bulk of a foreign body and/or suture material the better because it will be better tolerated, absorb faster, create less irritation and be felt less through the skin.
4. Stiffness/flexibility/malleability. The more malleable a foreign body, the better, especially in skin and subcutaneous tissue where there may be movement of the tissue relative to the foreign body.
5. Skin (and its derivative: leather) is compact and therefore able to be sutured/stitched. The subcutaneous tissue and its fat has a relatively sparse network of fibers and consequently is soft and loose and does not lend itself well to suturing. Because of the marked difference in density between the skin and the subcutaneous tissue, the deep surface of the skin acts as a barrier that restrains/checks/contains, secures and provides an attachment point to any retention device as well as prevent its extrusion.

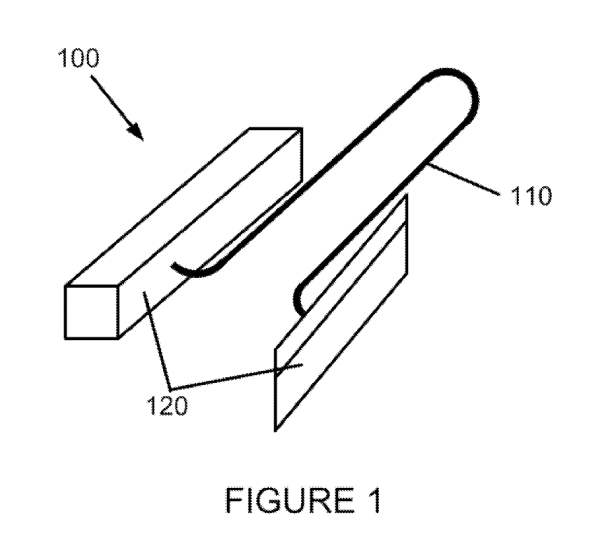

The invention will now be described making reference to the following drawings in which like reference numbers denote like structure or steps. As is shown in FIG. 1, a compound suture device 100, and preferably comprises a suture fiber 110 anchored by one or more solid retention members 120. Suture fiber 110 may be formed as a multifilament or monofilament fiber, and may further include braided multiple coated layers, smooth or textured and possess various types of indentations or projections pores and/or interstices or other appropriate material. Gluing welding, chemically bonding, tying, adhering, binding, cementing, linking, affixing, textile industry fabrication, or otherwise attaching material to itself may also be employed, and more than one suture may be employed. Any configuration or material may be acceptable as long as they possess the necessary characteristics of tensile strength and minimal bulk/volume. In the embodiment depicted in FIG. 1, two such retention members 120 are depicted, but one (or multiple) retention member may be employed, thus resulting in each end of suture fiber 110 being attached to the same and/or different retention member 120. Retention members 120 may be formed of the same or different material as suture fiber 110, and are preferably formed in a bulkier and/or stiffer form and/or different shape to properly anchor suture fiber 110, and to therefore retain the suture fiber in place. Thus, a unitary construction of the retention members and the suture or a non-unitary construction of the device may be employed. . Gluing welding, chemically bonding, tying, adhering, binding, cementing, linking, affixing, textile industry fabrication, or otherwise attaching material to itself may also be employed, and more than one suture may be employed when attaching the suture or sutures to the retention member or members or when constructing the retention member or members. Any method of construction and/or attachment is acceptable. Welded double or other multiple loop structure may be employed in order to form the retention members, thus using one or more pieces of the suture material together to form one or more of the retention members. These loops may be maintained, or may be cut to provide a flatter retention member. The looped material may be braided, glued, heat welded, chemically bonded, fabricated as in the textile industry or otherwise attached, and may be formed of a single or multiple loops, thus providing a variety of desirable shapes, such as a rectangular shape, cylindrical shape, or any other shape, and may further provide more robust, stiffer retention members. Any shape, consistency, chemical and physical composition, structure or material is acceptable as long as it anchors the suture to the undersurface of the skin. The same, or different material may be employed for the suture fiber and retention members, preferably providing material that absorbs more easily in the dermis layer for the suture fiber, while preferably providing material that absorbs more easily in a subcutaneous layer for the retention members, all while still retaining sufficient strength to form and/or be retained by the retention members. Absorbable, partially absorbable and non absorbable materials are also suitable for the suture, sutures retention member or members. If the suture is sufficiently robust, a single layer of this same material may be employed to form the retention members. As is shown in FIG. 1, suture fiber 110 is preferably formed with an oblong shape, but any desired shape may be employed in accordance with a particular incision shape or other characteristic. Suture fiber may further be formed in a partially rigid fashion, or may be formed malleable to be placed into a desired position by the user. Retention members 120 may similarly be formed in a partially rigid, fully rigid, or malleable format. Thus, a needle shape, harpoon shape, or any other methods, such as those known from clothing, textile, hang tagging, may be employed. Furthermore, they may provide an anchor to suture or sutures fiber at any point of each (if more than one) retention member 120.

The suture and retention members may be of unitary or non-unitary construction. They may be heat-welded, chemically bonded, glued, weaved, wrapped, looped, fabricated as in the various methods employed in the fabric industry, tied, coated, welded, adhered, binded, cemented, linked, affixed, textile industry fabricated, otherwise attached to itself, or otherwise attached. Any novel or conventional attachment method is acceptable as long as it maintains sufficient tensile strength necessary over the required period of time necessary. Any material or materials, any shape or shapes, any consistency or consistencies and any acceptable attachment method of the suture or sutures to the retention member or members is acceptable in the case of a unitary or non-unitary construction and will therefore fall within the scope of the present invention.

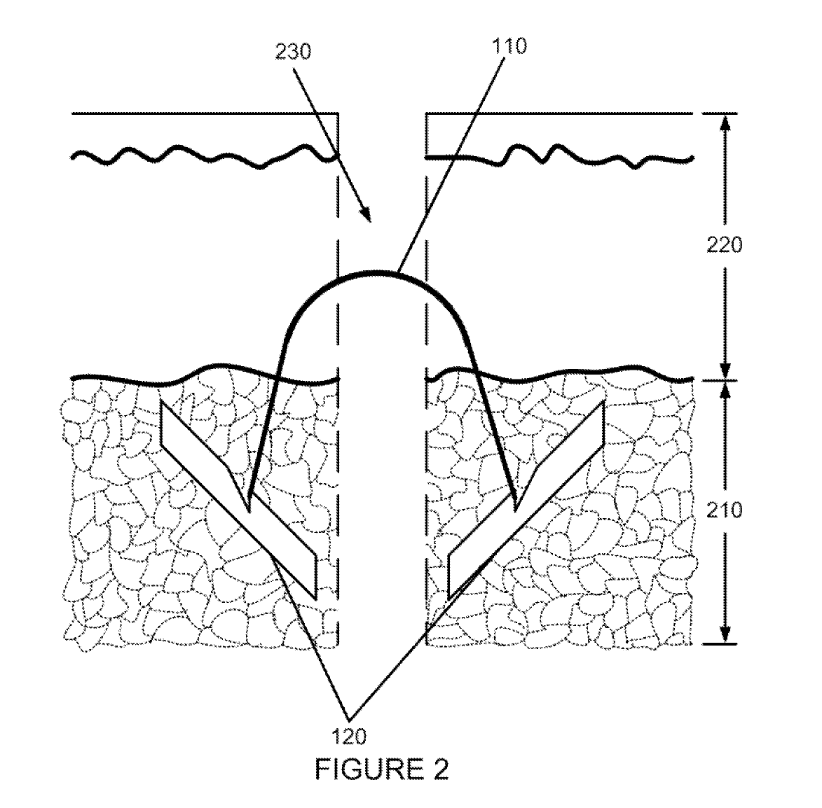

Referring next to FIG. 2, compound suture device 100 is shown embedded in a skin layer. As is shown in FIG. 2, each retention member 120 is preferably embedded in a subcutaneous fat layer 210 to anchor suture fiber 110. As is shown, suture fiber 110 preferably passes up from subcutaneous layer 210 through dermis-epidermis layer 220 where it approximates these dermis-epidermis layers of an incision 230 formed in skin 220 and subcutaneous fat 210. In such a manner, proper anchoring of the suture device can be provided in the subcutaneous layer, where absorption can take place, while non-bulky and/or more flexible approximating of the edges of the skin can take place in the dermis layer.

It will thus be seen that the objects set forth above, among those made apparent from the preceding description, are efficiently attained and, because certain changes may be made in carrying out the above method and in the construction(s) set forth without departing from the spirit and scope of the invention, it is intended that all matter contained in the above description and shown in the accompanying drawings shall be interpreted as illustrative and not in a limiting sense.

It is also to be understood that following claims are intended to cover all of the generic and specific features of the invention herein described and all statements of the scope of the invention which, as a matter of language, might be said to fall there between.

What is claimed:

1. A suture device for holding together and approximating skin edges, comprising:
one or more sutures; and
one or more retention members.

2. The suture device of claim 1, wherein the one or more sutures are formed of an absorbable suture material.

3. The suture device of claim 2, wherein the absorbable suture material is adapted to be substantially absorbed in a dermis layer of skin.

4. The suture device of claim 3, wherein the one or more sutures are formed of a malleable material.

5. The suture device of claim 3, wherein the one or more sutures are formed of a partially malleable material.

6. The suture device of claim 3, wherein the one or more sutures are formed of a partially rigid material.

7. The suture device of claim 3, wherein the one or more sutures are formed of a rigid material.

8. The suture device of claim 1, wherein the one or more sutures are formed of a partially absorbable material.

9. The suture device of claim 8, wherein the one or more sutures are formed of a malleable material.

10. The suture device of claim 8, wherein the one or more sutures are formed of a partially malleable material.

11. The suture device of claim 8, wherein the one or more sutures are formed of a partially rigid material.

12. The suture device of claim 8, wherein the one or more sutures are formed of a rigid material.

13. The suture device of claim 1, wherein the one or more sutures are formed of a non-absorbable material.

14. The suture device of claim 13, wherein the one or more sutures are formed of a malleable material.

15. The suture device of claim 13, wherein the one or more sutures are formed of a partially malleable material.

16. The suture device of claim 13, wherein the one or more sutures are formed of a partially rigid material.

17. The suture device of claim 13, wherein the one or more sutures are formed of a rigid material.

18. The suture device of claim 1, wherein each of the one or more retention members is formed of an absorbable material.

19. The suture device of claim 18, wherein the absorbable material is adapted to be substantially absorbed in a subcutaneous layer.

20. The suture device of claim 19, wherein each of the one or more retention members is formed of a malleable material.

21. The suture device of claim 19, wherein each of the one or more retention members is formed of a partially malleable material.

22. The suture device of claim 19, wherein each of the one or more retention members is formed of a partially rigid material.

23. The suture device of claim 19, wherein each of the one or more retention members is formed of a rigid material.

24. The suture device of claim 1, wherein each of the one or more retention members is formed of a partially absorbable material.

25. The suture device of claim 24, wherein each of the one or more retention members is formed of a malleable material.

26. The suture device of claim 24, wherein each of the one or more retention members is formed of a partially malleable material.

27. The suture device of claim 24, wherein each of the one or more retention members is formed of a partially rigid material.

28. The suture device of claim 24, wherein each of the one or more retention members is formed of a rigid material.

29. The suture device of claim 1, wherein each of the one or more retention members is formed of a non-absorbable material.

30. The suture device of claim 29, wherein each of the one or more retention members is formed of a malleable material.

31. The suture device of claim 29, wherein each of the one or more retention members is formed of a partially malleable material.

32. The suture device of claim 29, wherein each of the one or more retention members is formed of a partially rigid material.

33. The suture device of claim 29, wherein each of the one or more retention members is formed of a rigid material.

34. The suture device of claim 1, wherein the one or more retention members directly anchor the one or more sutures.

35. The suture device of claim 1, wherein the one or more retention members indirectly anchor the suture.

36. A suture device, comprising:
one or more sutures;
one or more suture retention members, each suture retention member anchoring a portion of a fiber of the one or more sutures.

37. The suture device of claim 1, wherein the bulk of the one or more suture retention members exceeds the bulk of the one or more sutures.

38. The suture device of claim 1, wherein the one or more sutures and the one or more suture retention members-are formed of a unitary structure.

39. The suture device of claim 1, wherein the one or more sutures and the one or more suture retention members are formed of a non-unitary construction.

40. The suture device of claim 1, wherein the suture device is confined deep to the outer surface of the skin.

41. The-suture device of claim 1, wherein the suture device is adapted to hold together and approximate skin edges.

42. The suture device of claim 1, wherein the suture device is situated both within the skin and subcutaneous tissue.

43. The suture device of claim 1, the absorbable suture device being anchored to the undersurface of one or more of the skin and the subcutaneous layer.
